(19) United States
(12) Patent Application Publication
(10) Pub. No.: US 2021/0321613 A1
(43) Pub. Date: Oct. 21, 2021

(54) Process for Preparation of Thiocyclam Base and Salt

(71) Applicant: ARYSTA LIFESCIENCE INC, Cary, NC (US)

(72) Inventors: Kamal Kataria, Kalyan (IN); Vic Prasad, Leawood, KS (US); Cameron Gibb, Apex, NC (US); Christopher Lynn Larson, Cary, NC (US)

(21) Appl. No.: 17/251,005

(22) PCT Filed: Jun. 12, 2019
(86) PCT No.: PCT/US2019/036851

§ 371 Date: Dec. 10, 2020

Related U.S. Application Data
(60) Provisional application No. 62/684,530, filed on Jun. 13, 2018.

(51) Int. Cl. A01N 43/32; C07D 341/00

(52) U.S. Cl. CPC A01N 43/32; C07D 341/00

(57) ABSTRACT

A process for preparation of thiocyclam base and salt. A method of manufacturing N,N-dimethyl-1,2,3-trithian-5-ylainine hydrochloride is described including the steps of: providing a mixture of thiosulfuric acid S, S′[2-(dimethylamino) trimethylene] ester monosodium salt and sodium hydroxide; adding an aqueous saline solution to the mixture of thiosulfuric acid S, S′-[2-(dimethylamino) trimethylene] ester monosodium salt and sodium hydroxide; separating the phases of the mixture; collecting the solids from the mixture using filtration; washing the solids with organic solvent; and drying the solids, where the dried solids are methyl-1,2,3-trithian-5-ylamine hydrochloride, and where the yield of the N,N-dimethyl-1,2,3-trithian-5-ylamine hydrochloride is increased by incorporation of a body charge of diatomaceous earth into the saline addition step, forming a mixture of the diatomaceous C earth with the composition of this step, and filtering the charged mixture through a diatomaceous earth media prior to the separation step.

### CROSS-REFERENCE TO RELATED APPLICATIONS

This application claims the benefit under 35 U.S.C. § 119(e) of U.S. Provisional Application No. 62/684,530 filed Jun. 13, 2018, which is related to co-pending, commonly assigned U.S. patent application Ser. No. 15/268,734, filed Sep. 19, 2016, entitled. MANUFACTURING METHOD FOR AND INSECTICIDAL COMPOSITIONS COMPRISING THIOCYCLAM HYDROCHLORIDE, the disclosures of which are expressly incorporated by reference herein in their entireties

### TECHNICAL FIELD

The field of art to which this invention generally pertains is insecticidal compositions.

### BACKGROUND

Insecticidal compositions, including pesticides, are typically formulated to kill, harm, repel or mitigate one or more species of insect. Insecticides work in different ways. Some insecticides disrupt the nervous system, whereas others may damage their exoskeletons, repel them or control them by some other means. They can also be packaged in various forms including sprays, dusts, gels, and baits. Because of these factors, each insecticide can pose a different level of risk to non-target insects, people, pets and the environment. Insecticides can be classified in two major groups: systemic insecticides, which have residual or long term activity; and contact insecticides, which have no residual activity. Systemic insecticides become incorporated and distributed systemically throughout the whole plant. When insects feed on the plant, they ingest the insecticide. Systemic insecticides produced by transgenic plants are called plant-incorporated protectants. Systemic insecticides have activity pertaining to their residue which is called “residual activity” or long-term activity. Contact insecticides are toxic to insects upon direct contact. These insecticides commonly fall into three categories. First, there are natural insecticides, such as nicotine, pyrethrum and neem extracts, made by plants as defenses against insects. Second there are inorganic insecticides, which are metals such as arsenates, copper and fluorine compounds. Third are organic insecticides, which are organic chemical compounds, typically working by direct contact with the insect or eggs and larvae. Insecticides are applied in various formulations and delivery systems such as sprays, baits, and slow-release diffusion. Efficacy can be related to the quality of pesticide application, with small droplets, such as aerosols often improving performance. Current treatments for controlling insects typically include chemicals, biologicals, and/or non-Chemical methods such as systemic acquired resistance inducers to provide resistant crop strains, GMO's, and hatching stimulants and inhibitors clear loci prior to planting.

Because of the chemical complexity of such insecticidal compositions, there is a constant challenge to generate effective insecticidal compositions at both purity levels and yields that make them commercially viable.

### BRIEF SUMMARY

A method of manufacturing N,N-dimethyl-1,2,3-trithian-5-ylainine hydrochloride is described including the steps of providing a mixture of thiosulfuric acid S, S′-[2-(dimethylamino) trimethylene] ester monosodium salt and sodium hydroxide, adding an aqueous saline solution to the mixture of thiosulfuric acid S, S′-[2-(dimethylamino) trimethylene] ester monosodium salt and sodium hydroxide, separating the phases of the mixture, collecting the solids from the mixture using filtration, washing the solids with organic solvent and drying the solids, where the dried solids are N,N-dimethyl-1,2,3-trithian-5-ylamine hydrochloride, and where the yield of the N,N-dimethyl-1,2,3-trithian-5-ylaminehydrochloride is increased by adding diatomaceous earth into the second step recited above, forming a mixture of the diatomaceous earth with the composition in this second step, and filtering the mixture through a diatomaceous earth media prior to the third step recited above.

Additional embodiments include: the method described above where the organic solvent comprises methyl-t-butyl ether, toluene, isopropanol or mixtures thereof; the method described above where the aqueous saline solution comprises sodium chloride; the method described above where the temperature during step b) is between −10° C. and −25° C.; the method described above where the temperature during the second step is between −15° C. and −20° C.; the method described above where 2M ethereal hydrochloric acid is added to the mixture after the third step and before the fourth step; the method described above where 2M hydrochloric acid in isopropanol is added to the mixture after the third step and before the fourth step; the method described above where the aqueous saline solution comprises sodium sulfide; the method described above where the purity of the N,N-dimethy 1,2,3-trithian-5-ylamine hydrochloride is greater than 90%; the method described above where the purity of the /N,N-dimethyl-1,2,3-trithian-5-ylamine hydrochloride is greater than 95%; the method described above where the % yield of N,N-dimethyl-1,2,3-trithian-5-ylamine hydrochloride is greater than 60%; the method described above where the concentration of HCl in the isopropanol is 2M to 5M; the method described above where dry hydrochloric acid gas is used in place of the hydrochloric acid and isopropanol, resulting in additionally increased yield; the method described above run as a batch process; the method described above run as a continuous process; and the method described above where the first three steps c are performed in an annular centrifugal reactor.

These, and additional embodiments, will be apparent from the following descriptions.

### DETAILED DESCRIPTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

The method of manufacturing thiocyclam hydrochloride is described in the reaction scheme below and in the following examples. Surprisingly high yields were able to be achieved with the modifications to the process described herein, while still maintaining high purity of the insecticidal compound. High purity could not previously be achieved in production methods of thiocyclam hydrochloride and therefore its use in insecticidal compositions had not been found effective compared to other known insecticidal compounds. But even with this high purity attained, increasing yield levels still presented meaningful challenges. One of the reasons for the modest yields is believed to be the result of emulsions formed during the work-up which leads to extended exposure to aqueous base and degradation prior to salt formation.

As described herein, the yield of the product is increased to greater than 60% by incorporation of a body charge (added to the body of the batch prior to filtering) of diatomaceous earth (e.g., CELITE® materials, Imerys Minerals California, Inc.) and subsequent filtration through the same diatomaceous earth material followed by the standard isolation procedure. The concentration of HCl in isopropanol used in the method described herein may vary from 2M (molar) to 5M without impacting purity or yield. The amount of diatomaceous earth added is can be up to 2 times the amount of monosultap present in the reaction mixture on a weight basis, for example, up to 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0. More can be used beyond 2 times, but the yield improvements would not be significant.

The yield can be further enhanced to greater than 90% using dry HCl gas in place of HCl/isopropanol, i.e. in toluene only. In both cases the purity is maintained above 95%. Dry HCl may be used for salt formation on thiocyclam free base resulting from batch or continuous processing.

In addition, a continuous process for manufacture of thiocyclam free base employing an annular centrifugal reactor (ACR) followed by HCl salt formation as described herein was also successfully demonstrated for preparation of thiocyclam hydrochloride. Using this apparatus aqueous solutions of monosultap/sodium chloride/sodium hydroxide and sodium sulfide/sodium chloride and toluene are continuously fed into the ACR, reaction is immediate within the ACR with subsequent rapid phase separation to protect the product from aqueous base. The purity is maintained above 95%, although the yield is modest (45-60%). There is the potential for enhanced efficiency and reduction in waste generation using the ACR. The ACR used with this embodiment is conventional ACR apparatus used in a conventional manner, for example available from Technoforce Solutions (I) Pvt Ltd.

The process for the hydrochloride may reasonably be extended to all acid salts of thiocyclam as well.

The basic reaction scheme for forming the thiocyclam hydrochloride described herein is as follows:

### EXAMPLE 1

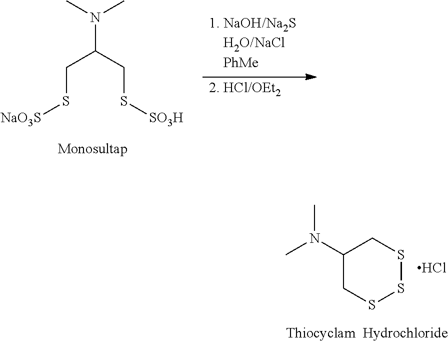

An aqueous saline solution of Na₂S.9H₂O (17.1 g) was added to a stirring mixture of monosultap (25 g), sodium hydroxide (2.86 g) and toluene/saline over 4.5 hours maintaining the temperature at −16° C. to −18° C. The reaction mixture was stirred at −16° C. to −18° C. until complete then filtered to remove inorganic salts. The cake was washed with toluene and combined with the filtrates. The phases were separated and the toluene solution was washed with water then brine and dried over sodium sulfate. 2M ethereal hydrochloric acid (50 mL (millilitres)) was added and the mixture was stirred for 1 hour. The solids formed were collected by filtration, washed with MTBE (methyl tart-butyl ether) and dried to give Thiocyclam Hydrochloride in 87% yield (14.2 g) and 97.9% purity with 0.36% residual toluene. However, it would be difficult at best to use ethereal hydrochloric acid outside a laboratory environment, for example, in a commercial environment.

### EXAMPLE 2

An aqueous saline solution of Na₂S.9H₂O (15.3 g) was added to a stirring mixture of monosultap (25 g), sodium hydroxide (3.16 g) and toluene/saline over 4 hours maintaining the temperature at −15° C. to −18° C. The reaction mixture was stirred at −15° C. until complete then filtered to remove inorganic salts. The cake was washed with toluene and the wash was combined with the filtrates. The phases were separated and the toluene solution was washed with water then brine and dried over sodium sulfate. 2M ethereal hydrochloric acid (50 mL) was added and the mixture was stirred for 1 hour. The solids formed were collected by filtration, washed with ice-cold MTBE and dried to give Thiocyclam Hydrochloride in 76% yield (12.4 g) and 98.3% purity bearing 0.22% residual toluene. However, it would be difficult at best to use ethereal hydrochloric acid outside a laboratory environment, for example, in a commercial environment.

### EXAMPLE3

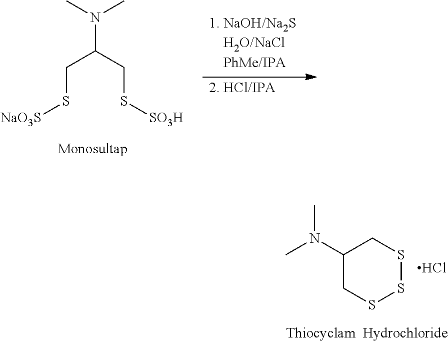

An aqueous saline solution of Na₂S.9H₂O (92.6 g) was added to a stirring mixture of monosultap (100 g), sodium hydroxide (11.3 g) and toluene/saline/isopropanol over 5.5 hours maintaining the temperature at −20° C. The reaction mixture was stirred at −20° C. until complete then the phases were separated. The toluene solution was washed with water then brine and dried over sodium sulfate. 2M hydrochloric acid in isopropanol (224 mL) was added to the batch over 30 minutes and the mixture was stirred for 1 hour. The solids formed were collected by filtration, washed with isopropanol and dried to give thiocyclam hydrochloride in 60% yield (37.0 g) and 95.6% purity.

### EXAMPLE 4

A mixture of monosultap (450 g), sodium hydroxide (51.3 g) and saline was added to a reactor containing toluene at −15° C. to −20° C. An aqueous saline solution of Na₂S.9H₂O (308 g) was added to the batch over 4 hours maintaining the temperature at −15° C. The reaction mixture was stirred at −15° C. until complete then the phases were separated. After an aqueous work-up, the organics were dried over magnesium sulfate and 2M ethereal hydrochloric acid (710 mL) was added. The mixture was stirred for 1 hour then the solids formed were collected by filtration. The cake was washed with toluene and dried to give thiocyclam hydrochloride in 68% yield (214 g) and 94.2% purity containing 0.55% residual toluene. However, it would be difficult at best to use ethereal hydrochloric acid outside a laboratory environment, for example, in a commercial environment.

### EXAMPLE 5

An aqueous saline solution of Na₂S.9H₂O (239.5 g) was added to a stirring mixture of monosultap (350 g), sodium hydroxide (39.9 g) and toluene/saline over 3.5 hours maintaining the temperature at −16° C. to −18° C. The reaction mixture was stirred at −16° C. to −18° C. until complete then filtered to remove inorganic salts. The cake was washed with toluene and the wash was combined with the filtrates. The phases were separated and the toluene solution was washed with water then brine and dried over sodium sulfate. 2M ethereal hydrochloric acid (680 mL) was added to the batch over 30 minutes and the mixture was stirred for 1 hour. The solids formed were collected by filtration, washed with ice-cold MTBE and dried to give thiocyclam hydrochloride in 33% yield (76.1 g) and 95.2% purity, with 0.05% residual toluene.

### EXAMPLE 6

An aqueous saline solution of Na₂S.hydrate (53.05 g) was added to a stirring mixture of monosultap (100 g), sodium hydroxide (10.8 g) and toluene/saline/isopropanol over 1.5 hours maintaining the batch temperature at less than −15° C. The reaction mixture was stirred at −20° C. to −15° C. until complete. CELITE material (100 g) was charged to the mixture and stirring was continued at −20° C. to −15° C. for 10 minutes. The hatch was filtered through a pad of CELITE material and the filter cake was washed with toluene. The combined filtrates were separated and the organics were washed with water then brine and dried over sodium sulfate. 2M hydrochloric acid in isopropanol (213 mL) was added to the batch over 30 minutes and the mixture was stirred for 1 hour. The solids formed were collected by filtration, washed with toluene and dried to give thiocyclam hydrochloride in 80% yield (50.0 g) and 97.6% purity.

### EXAMPLE 7

An aqueous saline solution of Na: S-hydrate (26.5 g) was added to a stirring mixture of Monosultap (50 g), sodium hydroxide (5.4 g) and toluene/saline/isopropanol over 1.5 hours maintaining the batch temperature at less than −15° C. The reaction mixture was stirred at −20° C. to −15° C. until complete. CELITE material (50 g) was charged to the mixture and stirring was continued at −20° C. to 15° C. for 10 minutes. The batch was filtered through a pad of CELITE material and the filter cake was washed with toluene. The combined filtrates were separated and the organics were washed with water then brine and dried over sodium sulfate. 5M hydrochloric acid in isopropanol (4.2 mL) was added to the batch over 30 minutes and the mixture was stirred for 1 hour. The solids formed were collected by filtration, washed with toluene and dried to give thiocyclam hydrochloride in 82% yield (25.5 g) and 97.9% purity.

### EXAMPLE 8

An aqueous saline solution of Na₂S.hydrate (53.05 g) was added to a stirring mixture of monosultap (100 g), sodium hydroxide (10.8 g) and toluene/saline/isopropanol over 1.5 hours maintaining the batch temperature at less than −15° C. The reaction mixture was stirred at −20 to −15° C. until complete. CELITE material (100 g) was charged to the mixture and stirring was continued at −20 to −15° C. for 10 minutes. The batch was filtered through a pad of CELITE material and the filter cake was washed with toluene. The combined filtrates were separated and the organics were washed with water then brine and dried over sodium sulfate. HCl gas was bubbled through the batch over 2.5 hours and the resultant mixture was stirred for 16 hours. The solids formed were collected by filtration, washed with toluene and dried to give thiocyclam hydrochloride in 88% yield (56.0 g) and 95.4% purity.

### EXAMPLE 9

Solution A: Monosultap (200 g) and sodium hydroxide (21.6 g) were dissolved in saline, the solution was filtered to remove particulates and then cooled to 0° C.

Solution B: Sodium sulfide (106.1 g) was dissolved in saline, the solution was filtered to remove particulates and then cooled to 5° C.

Reaction: The ACR was pre-cooled to −20° C. then solution A, solution B and toluene (pre-cooled to −20° C.) were charged to the reactor via conventional pumps over 30 minutes. The organic phase was collected from the outlet of the ACR in fractions and analysed for purity by GC (gas chromatography).

| Fraction | 1 | 2 | 3 | 4 | 5 | 6 | 7 | Average |
|---|---|---|---|---|---|---|---|---|
| Thiocyclam | 96.48 | 96.74 | 96.41 | 95.75 | 93.77 | 97.0 | 94.85 | 95.85 |
| free-base | | | | | | | | |
| (%) | | | | | | | | |

In some embodiments, there are provided insecticidal compositions comprising thiocyclam hydrochloride, as synthesized by the processes described herein, where the lethal dose and lethal concentration of active insecticide is much lower than previously studied insecticides, therefore making thiocyclam hydrochloride an effective insecticide. These compositions are particularly useful in the elimination of target insects on agricultural crops. Target sucking insects may include mosquitoes (for example Aedes aegypti, Aedes vexans, Culex quinquefasciatus, Culex tarsalis, Anopheles albimanus, Anopheles stephensi, Mansonia titillans), moth gnats (for example Phlebotomus papatasii), gnats (for example Culicoides furens), buffalo gnats (for example Simulium damnosum), stinging flies (for example Stomoxys calcitrans), tsetse flies (for example Glossina morsitans morsitans), horse flies (for example Tabanus nigrovittatus, Haematopota pluvialis, Chrysops caecutiens), true flies (for example Musca domestica; Musca autumnalis, Musca vetustissima, Fannia canicularis), flesh flies (for example Sarcophaga carnaria), myiasis-causing flies (for example Lucilia cuprina, Chrysomyia chloropyga, Hypoderma bovis, Hypoderma lineatum, Dermatobia hominis, Oestrus ovis, Gasterophilus intestinalis, Cochliomyia hominivorax), bugs (for example Cimex lectularius, Rhodnius prolixus, Triatoma infestans), lice (for example Pediculus humanis, Haematopinus suis, Damalina ovis), fleas (for example Pulex irritans, Xenopsylla cheopis, Ctenocephalides canis, Ctenocephalides felis), and sand fleas (Tunga penetrans).

The current composition is especially effective for eliminating aphids and white flies. Additional target species are Lepidoptera (moths and butterflies), which is the second largest order in the class Insecta. Nearly all lepidopteran larvae are called caterpillars. They have a well-developed head with chewing mouthparts. In addition to three pairs of legs on the thorax, they have two to eight pairs of fleshy abdominal prolegs that are structurally different from the thoracic legs. Most lepidopteran larvae are herbivores; some species eat foliage, some burrow into stems or roots, and some are leaf-miners. The composition and method of the present invention were found to be particularly advantageous for use in the control of insects in crops. Suitable target crops include, for example, cereals, including wheat, barley, rye, oats; rice, maize, sorghum, millet and manioc; beets, including sugar beets and fodder beets; fruits, including ponies, stone fruit and soft fruit, such as apples, pears, plums, peaches, almonds; cherries, or berries, for example strawberries, raspberries and blackberries; leguminous plants, including beans, lentils, peas and soybeans; oil plants, including rape, mustard, poppy, olives, sunflowers, coconut, castor oil plants, cocoa beans and groundnuts; cucurbitaceae, including marrows, cucumbers and melons; fibrous plants, including cotton, flax, hemp and jute; citrus fruit, including oranges, lemons, grapefruit and mandarins; vegetables, including spinach, lettuce, asparagus, cabbages, carrots, onions, tomatoes, potatoes and paprika; lauraceae, including avocados, cinnamon and camphor; as well as tobacco; nuts, coffee, aubergines, sugar cane; tea, pepper, vines, hops; bananas, natural rubber plants, eucalyptus, and ornamental plants. Examples of some preferred crops for insecticidal treatment include radish and beans.

As used herein, the term “insecticide” refers to a compound used to control (including prevention, reduction or elimination) parasitic insects. “Controlling insects” as used in the present invention means killing insects or preventing insects from developing or growing. Controlling insects as used herein also encompasses controlling insect progeny (development of viable cysts and/or egg masses). The compound described herein, may be used to keep an agricultural crop healthy and may be used curatively, preventively or systematically to control insects, “Agricultural crops” as described herein, may refer to a wide variety of agricultural plants. When using the compounds described herein to keep a plant healthy, the controlling of insects includes the reduction of damage to plants and increased yield of the crop. The current invention achieves this endeavor by efficiently ridding a plant of insect pests by using a low concentration of the insecticidal composition to rid the crop of larger populations of insects than previous insecticides could eliminate. Insecticidal effects typically relate to diminishing the occurrence or activity of the target insect. Such effects on the insect include necrosis, death, retarded growth, diminished mobility, lessened ability to remain on the host plant, reduced feeding and inhibition of reproduction. These effects on insects provide control (including prevention, reduction or elimination) of parasitic infestation of the plant. Therefore the term “control” of a parasitic insect means achieving a pesticidal effect on the insect.

The expressions “insecticidally effective amount” and “biologically effective amount” in the context of applying a chemical compound to control a parasitic insect refer an amount of the compound that is sufficient to protect an agricultural crop from destruction by such insects. In embodiments herein, the total content of components in the insecticidal composition is 100 weight percent. The insecticidal compositions of the present invention may further contain one or more agriculturally acceptable auxiliaries. The auxiliaries employed in the insecticidal composition and their amounts will depend in part upon the type of formulation or composition and/or the manner in which the formulation is to be applied. Suitable auxiliaries include, but are not limited to formulation adjuvant or components, such as solvents, surfactants, stabilizers, anti-foaming agents, anti-freezing agents, defoamers, emulsifiers, preservatives, antioxidants, colorants, thickeners and inert fillers and these auxiliaries may be used individually in the agrochemical composition or as a combination of one or more auxiliaries. Auxiliaries may be present in the composition anywhere from 0.01-90 parts by weight. For example, the composition may comprise one or more solvents, which may be organic or inorganic.

Suitable solvents are those that thoroughly dissolve the agrochemically active substance employed. Examples of suitable solvents include water, aromatic solvents, such as xylene (for example solvent products commercially available from Solvesso™), mineral oils, animal oils, vegetable oils, alcohols, for example methanol, butanol, pentanol, and benzyl alcohol; ketones, for example cyclohexanone, and gamma-butyrolactone, pyrrolidones, such as NMP (N-methyl-2-pyrrolidone), and NOP, acetates, such as glycol diacetate, glycols, fatty acid dimethylamides, fatty acids, and fatty acid esters. The composition may optionally include one or more surfactants. Suitable surfactants are generally known in the art and include, but are not limited to, alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, arylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose and ethylene oxide/propylene oxide block copolymers.

The composition may optionally comprise one or more polymeric stabilizers. Suitable polymeric stabilizers that may be used in the present invention include, but are not limited to, polypropylene, polyisobutylene, polyisoprene, copolymers of monoolefins and diolefins, polyacrylates, polystyrene, polyvinyl acetate, polyurethanes or polyamides. The composition may include an anti-foaming agent. Suitable anti-foam agents include, for example, mixtures of polydimethylsiloxanes and perfluroalkylphosphonic acids, such as silicone anti-foam agents. One or more preservatives may also be present in the composition. Suitable examples include, for example, Preventol® material (commercially available from Bayer AG) and Proxel® material (commercially available from Bayer AG), Furthermore, the composition may also include one or more antioxidants, such as butylated hydroxytoluene. The compositions may further comprise one or more solid adherents. Such adherents are known in the art and available commercially. They include organic adhesives, including tackifiers, such as celluloses of substituted celluloses, natural and synthetic polymers in the form of powders, granules, or lattices, and inorganic adhesives such as gypsum, silica, or cement.

The compositions may include one or more inert fillers, including, for example, natural ground minerals, such as kaolins, aluminas, talc, chalk, quartz, attapulgite, montmorillonite, and diatomaceous earth, or synthetic ground minerals, such as highly dispersed silicic acid, aluminum oxide, silicates, and calcium phosphates and calcium hydrogen phosphates. Suitable inert fillers for granules include, for example, crushed and fractionated natural minerals, such as calcite, marble, pumice, sepiolite, and dolomite, or synthetic granules of inorganic and organic ground materials, as well as granules of organic material, such as sawdust, coconut husks, corn cobs, and tobacco stalks. The compositions may also include one or more thickeners, including, for example, gums, such as xanthan gum, polyvinyl alcohol, cellulose and its derivatives, clay hydrated silicates, magnesium aluminum silicates or a mixture thereof. The insecticidal composition further may include a safener. The safener, also called antidote, may comprise, at least one of isoxadifen-ethyl, 1,8-dicarboxylic anhydride, mefenpyr- diethyl, fenchlorazole-ethyl, and cloquintocet-mexyl, and in some embodiments, isoxadifenethyl. The dosage of the safener may be a conventional dosage used for matching the thiocyclam hydrochloride. In some embodiments, relative to 1 part by weight of thiocyclam hydrochloride, the safener has a content of from 0.1 to 10 parts by weight, or in some embodiments, from 0.5 to 5 parts by weight. In some embodiments of the present invention, the insecticidal composition may be applied and used in pure form, or more preferably together with at least one of the auxiliaries, as described above.

The composition of the present invention may also comprise other active ingredients for achieving specific effects, for example, bactericides, fungicides, nematicides, molluscicides or herbicides. Suitable compounds are known in the art. The insecticidal composition of the present invention may be formulated in different ways, depending upon the circumstances of its use. Suitable formulation techniques are known in the art and include water-dispersible powders, dusts, pastes, water-dispersible granules, solutions, emulsifiable concentrates, emulsion, suspension concentrate, aerosols, or microencapsulation suspensions.

Examples of formulation types for use in the present invention include the following: water-soluble concentrates, in which thiocyclam hydrochloride and/or solvate thereof is dissolved in a water-soluble solvent. One or more wetting agents and/or other auxiliaries may be included. The active compound dissolves upon dilution with water; emulsifiable concentrates, in which thiocyclam hydrochloride and/or solvate thereof is dissolved in a water-immiscible solvent, preferably with the addition of one or more non-anionic emulsifiers and anionic emulsifiers. The mixture is agitated, for example by stirring, to get a uniform formulation. Dilution with water provides a stable emulsion; emulsions, in which thiocyclam hydrochloride and/or solvate thereof is dissolved in one or more suitable water immiscible solvents, preferably with the addition of one or more non-anionic emulsifiers and anionic emulsifiers. The resulting mixture is introduced into water by appropriate means, such as an emulsifying machine, to provide a homogeneous emulsion. Dilution with water gives a stable emulsion; suspensions, in which thiocyclam hydrochloride and/or solvate thereof is comminuted in an agitated ball mill, preferably with the addition of one or more dispersants and wetting agents, and water or solvent to give a fine active compound suspension.

Dilution with water gives a stable suspension of the active compound; water-dispersible granules and/or water-soluble granules in which thiocyclam hydrochloride and/or solvate thereof is ground finely, preferably with the addition of one or more dispersants and wetting agents, and prepared as water-dispersible or water-soluble granules by means of suitable techniques, for example by extrusion, drying in a spray tower, or by processing in a fluidized bed. Dilution with water gives a stable dispersion or solution of the active compound; water-dispersible powders and water-soluble powders, in which thiocyclam hydrochloride and/or solvate thereof is ground in a suitable apparatus, such as a rotor-stator mill, preferably with addition of one or more dispersants, wetting agents and silica gel. Dilution with water gives a stable dispersion or solution of the active compound; granules, in which thiocyclam hydrochloride and/or solvate thereof is finely ground in a suitable apparatus, with addition of up to 99.5 parts by weight of carriers. Granules can then be prepared either by suitable techniques, such as extrusion, spray-drying or using a fluidized bed.

In general, the composition or formulation is prepared and applied such that the insecticidal composition comprising thiocyclam hydrochloride and/or solvate thereof is applied at any suitable rate, as demanded by the insect to be treated. The application rate may vary within wide ranges and depends upon such factors as the type of application (i.e., foliar application, seed dressing, application in the seed furrow, etc.), the target crop plant, the particular insect(s) to be controlled, the climatic circumstances prevailing in each case, as well as other factors determined by the type of application, timing of application and target crop. Typically, the application rate may be from about 1 to about 2000 g of the insecticidal composition per hectare, and depending on the various factors described above, may be 10 to 1000 g/ha, more preferably 10 to 500 g/ha, more preferably 10 to 200 g/ha.

The use of the insecticidal composition or formulation comprising thiocyclam hydrochloride and/or solvate thereof may be applied at any suitable time. In some embodiments, the composition is applied the locus of the plant prior to planting, during planting, or after planting. Such a treatment may take place by conventional methods known in the art, including, for example, drip-irrigation, chem-irrigation, and spray. In one embodiment, the insecticidal composition is contacted with the plant, plant part, or a locus thereof immediately before or immediately after the plant is transplanted. For application to plant foliage, the insecticidal composition can be diluted up to about 600-fold or more with water, more typically up to about 100-fold or up to about 40-fold. Illustratively, a concentrate product can be applied at about 0.1 to about 30 liter/hectare (l/ha), for example about 5 to about 25 l/ha, in a total application volume after dilution of about 60 to about 600 l/ha, for example about 80 to about 400 l/ha or about 100 to about 200 l/ha. Other concentrations of the concentrate compositions disclosed herein can be used. One skilled in the art will recognize that a particular system may dictate a good working application rate which may depend in part on both the insects to be controlled as well as the crop requiring protection. The dosage range for the components of the inventive insecticidal composition allows for use of a reduced amount of active ingredient when used in the composition as described. The result is an inferior dosage rate that provides a more efficient insecticide. As used herein, the term “about” refers to a measurable value such as a parameter, an amount, a temporal duration, and the like and is meant to include variations of +/−15% or less, preferably variations of +1-10% or less, more preferably variations of +/−5% or less, even more preferably variations of +/−1% or less, and still more preferably variations of +1-0.1% or less of and from the particularly recited value, in so far as such variations are appropriate to perform in the invention described herein. Furthermore, it is also to be understood that the value to which the modifier “about” refers is itself specifically disclosed herein.

The use of the insecticidal composition comprising thiocyclam hydrochloride and/or solvate thereof for treating plants, plant parts, or a locus thereof is through the use of various processing methods carried out directly on the plant or plant parts or to the environment, the habitat or storage space of the plant or plant parts. These methods include, for example, dipping, spraying, atomizing, irrigation, evaporation, powdering, misting, fogging, spreading, foam, coating, painting, spreading-on, watering, soaking, drip irrigation, and chemirrigation. While certain forms of thiocyclam are known for use as a pesticide, the use of thiocyclam hydrochloride has not previously been successfully manufactured and therefore not contemplated for use as an insecticide. The reduced concentration and increased efficiency provided by the use of the synthesized thiocyclam hydrochloride were both surprising and unexpected. The following are non-limiting examples, wherein thiocyclam hydrochloride is compared to previously known insecticides, cartap and thiocyclam oxalate. These examples are merely illustrations and are not to be understood as limiting the scope and underlying principles of the invention in any way. Various modifications of the invention in addition to those shown and described herein will become apparent to those skilled in the art form after the following examples and foregoing description. Such modifications are also intended to fall within the scope of the appended claims.

### EXAMPLE 10

Coding moth, Cydia pomonella—onate

larvae
Bioassay by diet incorporation;
Mortality assessment 2 days after treatment
a.i. refers to active ingredient

| | | LC50 | | | LC90 | | |
|---|---|---|---|---|---|---|---|
| | Purity | (μg of | | | (pg of | | |
| | of | a.i/ml | | | a.i/ml | | |
| D + 2 | a.i. | of diet) | min | max | of diet) | min | max |
| Cartap | 50% | 10.14 | 7.98 | 12.87 | 104.80 | 74.72 | 157.80 |
| Thio- | 87.5% | 3.57 | 3.00 | 4.25 | 19.76 | 15.53 | 26.32 |
| cyclam | | | | | | | |
| oxalate | | | | | | | |
| Thio- | 100% | 2.57 | 2.14 | 3.09 | 10.80 | 8.41 | 14.79 |
| cyclam | | | | | | | |
| HCl | | | | | | | |

### EXAMPLE 11

Soybean looper, Chrysodeixis includens—3rd larval stage

Bioassay by diet surface incorporation
Mortality assessment 3 days after treatment
a.i. refers to active ingredient

| | | LC50 | | | | | |
|---|---|---|---|---|---|---|---|
| | Purity | (μg of | | | LC90 | | |
| | of | a.i/ml | | | (μg of | | |
| D + 3 | a.i. | of diet) | min | max | a.i./ml) | min | max |
| Cartap | 50% | 53,069 | 31,907 | 106,810 | 908,897 | 346,473 | 4.64 × 10⁶ |
| Thiocyclam | 87.5% | 173,363 | 52,529 | 2.38 × 10⁶ | 8.55 × 10⁶ | 906,914 | 1.53 × 10⁹ |
| oxalate | | | | | | | |
| Thiocyclam | 100% | 8,992 | 6,520 | 12,564 | 251,759 | 145,956 | 504,401 |
| HCl | | | | | | | |

### EXAMPLE 12

Aphids, Myzus persicae—larvae

Study based on IRAC n°019 methods, application method adapted
Plant: radish
Application on petri dish with larvae with a boom sprayer apparatus
Assessment of mortality 3 and 4 days after application
a.i. refers to active ingredient

| | Purity | LD50 | | | LD90 |
|---|---|---|---|---|---|
| D + 3 | of a.i. | (g a.iJha) | min | max | (g al./ha) |
| Cartap | 50% | <18.18 | NA | NA | 36.70 |
| Thiocyclam | 87.5% | 7.99 | 1.40 | 54.98 | 38.06 |
| oxalate | | | | | |
| Thiocyclam | 100% | 12.04 | 0.40 | 358.91 | 30.51 |
| HCI | | | | | |
| | Purity | LD50 | | | LD90 |
| D + 4 | of a.i. | (g al./ha) | min | max | (g alJha) |
| Cartap | 50% | <18.18 | NA | NA | 16 |
| Thiocyclam | 87.5% | 5.35 | 2.60 | 10.27 | 29.57 |
| oxalate | | | | | |
| Thiocyclam HC1 | 100% | 7.66 | 2.30 | 25.48 | 27.07 |

### EXAMPLE 13

White flies, Trialeurodes vaporarium—larvae

Study based on IRAC n°015 methods, stage and application method adapted
Plant: bean
Application: On the petri dish with larvae with a boom sprayer apparatus
Assessment of mortality at 7, 9 and 14 days after application
a.i. refers to active ingredient

| | Purity | LDS( ) | | | LD90 |
|---|---|---|---|---|---|
| D + 7 | of a.i. | (g a.iJha) | min | max | (g ai./ha) |
| Cartap | 50% | 213.7 | 0.76 | 2.23 × | 2.23 × |
| | | | | 10 | 10⁶ |
| Thiocyclam | 87.5% | 19.5 | 16.4 | 24.8 | 54.2 |
| oxalate | | | | | |
| Thiocyclam | 100% | 2.22 | 0.71 | 4.15 | 1,899 |
| HCI | | | | | |
| | Purity | LD50 | | | LD90 |
| D + 9 | of a.i. | (g al/ha) | min | max | (g a.i/ha) |
| Cartap | 50% | 15.6 | 0.027 | 8,923.2 | 4,566.3 |
| Thiocyclam | 87.5% | 15.3 | 1.6 | 147.6 | 138.3 |
| oxalate | | | | | |
| Thiocyclam | 100% | 2.23 | 0.12 | 38.9 | 4,905 |
| HCI | | | | | |
| | Purity | LD50 | | | LD90 |
| D + 114 | of a.i. | (g a.i./ha) | min | max | (g a.i./ha) |
| Cartap | 50% | 10.4 | 0.05 | 2,098.7 | 910.4 |
| Thiocyclam | 87.5% | 15.7 | 1.12 | 219 | 153.1 |
| oxalate | | | | | |
| Thiocyclam | 100% | 0.82 | 0.018 | 386.3 | 7,910 |
| HCI | | | | | |

LC stands for “Lethal Concentration”. LC values refer to the concentration of a chemical required to kill a certain proportion of a population of pests. The concentration of the chemical that kills 50 percent of the pests during the observation period is the LC50 value and a concentration that kills 90 percent of a population is LC90, LD stands for “Lethal Dose”. LD50 is the amount of an ingested substance that kills 50 percent of a test sample and LC90 is the lethal dose that kills 90 percent of the test sample. As can be seen from the results in the examples, thiocyclam hydrochloride with high purity manufactured using the methods described herein, so that a low concentration of thiocyclam hydrochloride can be used to effectively eliminate insects similar to or better than prior known insecticides. In example 1, approximately 4 times less thiocyclam hydrochloride was ingested by the moths at LC50 compared to cartap and approximately 9 times less at LC90 compared to cartap. The thiocyclam hydrochloride produced by the methods provided herein also achieved an LC50 and LC90 at significantly lower concentrations than thiocyclam oxalate. In example 2, the LC50 of the soybean looper was achieved with approximately 6 times less thiocyclam hydrochloride than cartap and approximately 20 times less thiocyclam hydrochloride than thiocyclam oxalate. The LC90 of the soybean looper was orders of magnitude smaller using thiocyclam hydrochloride compared to thiocyclam oxalate, and the LC90 for thiocyclam hydrochloride was more than three times lower than the concentration of cartap. In example 7, all three insecticides performed similarly to eliminate the aphids on radish plants. In example 8, the thiocyclam hydrochloride outperforms the other insecticides significantly for achieving LD50, on all assessment days. At all assessment days, the LD90 was achieved at the lowest concentration using thiocyclam oxalate. On days 7 and 9, thiocyclam hydrochloride achieved the LD90 at a lower application concentration compared to cartap. The use of the thiocyclam hydrochloride composition embodiments is in no way restricted to these genera, but also extends in the same manner to other insects and other crops. The novel high purity manufacturing method for thiocyclam hydrochloride has surprisingly allowed for improved insecticidal qualities when the thiocyclam hydrochloride is used in insecticidal compositions and applied to agricultural crops.

The purity levels of the improved method as disclosed herein are typically equal to or greater than about 90%, 91%, 92%, 93%, 94%, 95%, 96%, 97%, and 98%, and the yields equal to or greater than about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include aspects that are not expressly included in the invention are nevertheless disclosed herein. A number of embodiments of the invention have been described. Nevertheless, it will be understood that various modifications may be made without departing from the spirit and scope of the invention. In addition, it shall be pointed out that specific technical features described in the above specific embodiments, if reconcilable, can be combined in any appropriate manner, and in order to avoid unnecessary repetition, various possible combination manners are not otherwise stated herein any more.

As described herein, these problems and others in this area are addressed by the invention described herein. Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of manufacturing N,N-dimethyl-1,2,3-trithian-5-ylamine hydrochloride, comprising
a. providing a mixture of thiosulfuric acid S, S′-[2-(dimethylamino) trimethylene] ester monosodium salt and sodium hydroxide;
b. adding an aqueous saline solution to the mixture of a) to provide the N,N-dimethyl-1,2,3-trithian-5-ylamine hydrochloride;
c. adding diatomaceous earth into step b) thus forming a mixture of the diatomaceous earth with the composition of b); and
d. filtering the mixture of c) through a diatomaceous earth media;
wherein the yield of the N,N-dimethyl-1,2,3-trithian-5-ylamine hydrochloride is increased by adding diatomaceous earth in step c), and filtering the mixture through a diatomaceous earth media in d).

2. The method according to claim 1, further comprising
e. separating phases of the mixture of c);
f. collecting solids from e) using filtration;
g. washing the solids from f) with an organic solvent; and
h. drying the solids from g);
wherein the dried solids comprises N,N-dimethyl-1,2,3-trithian-5-ylamine hydrochloride.

3. The method according to claim 2, wherein the organic solvent comprises methyl-t-butyl ether, toluene, isopropanol, or a mixtures thereof.

4. The method according to claim 1, wherein the aqueous saline solution comprises sodium chloride.

5. The method according to claim 1, wherein the temperature during step b) is between 10° C. and −25° C.

6. The method according to claim 1, wherein the purity of the N,N-dimethyl-1,2,3-trithian-5-ylamine hydrochloride is greater than 90%.

7. The method according to claim 6, wherein the purity of the N,N-dimethyl-1,2,3-trithian-5-ylamine hydrochloride is greater than 95%.

8. N,N-dimethyl-1,2,3-trithian-5-ylamine hydrochloride having a purity greater than 90%.

9. An insecticidal composition comprising the N,N-dimethyl-1,2,3-trithian-5-ylamine hydrochloride of claim 8, wherein the effective dose of N,N-dimethyl-1,2,3-trithian-5-ylamine hydrochloride having purity of greater than 90% is substantially reduced in comparison to N,N-dimethyl-1,2,3-trithian-5-ylamine hydrochloride having purity lesser than 90%.

10. A method for controlling insect pests at a locus, said method comprising applying, to the locus, an amount from about 1 to about 2000 g of a composition comprising N,N-dimethyl-1,2,3-trithian-5-ylamine hydrochloride having a purity greater than 90%, wherein the effective dose of N,N-dimethyl-1,2,3-trithian-5-ylamine hydrochloride having purity greater than 90% is substantially reduced in comparison to the compound having purity lesser than 90%.

11. The method according to claim 10, wherein said composition is diluted up to about 600-fold or more with water before application.

12. The method according to claim 11, wherein the composition is applied at about 0.1 to about 30 liter/hectare in a total application volume after dilution of about 60 to about 600 L/ha.

13.

14. The method according to claim 10, wherein the LC50 of the N,N-dimethyl-1,2,3-trithian-5-ylamine hydrochloride is between about 2.0 to about 3.50 μg of a.i./mL, or wherein the LC90 of the N,N-dimethyl-1,2,3-trithian-5-ylamine hydrochloride is between about 8.0 to about 15.00 μg of a.i./mL at 100% purity of the N, N-dimethyl-1,2,3-trithian-5-ylamine hydrochloride.

15. The method according to claim 10, wherein the LC50 of the N,N-dimethyl-1,2,3-trithian-5-ylamine hydrochloride is between about 6.5 to about 13.00 μg of a.i./mL, or wherein the LC90 of the N,N-dimethyl-1,2,3-trithian-5-ylamine hydrochloride is between about 100.0 to about 550.0 μg of a.i./mL at 100% purity of the N,N-dimethyl-1,2,3-trithian-5-ylamine hydrochloride.

16. The method according to claim 10, wherein the LC50 of the N,N-dimethyl-1,2,3-trithian-5-ylamine hydrochloride is about 12.0 g of a.i./ha, or wherein the LC90 of the compound is 30.00 g of a.i./ha at 100% purity of the N,N-dimethyl-1,2,3-trithian-5-ylamine hydrochloride.

17. The method according to claim 10, wherein the LC50 of the N,N-dimethyl-1,2,3-trithian-5-ylamine hydrochloride is between about 7.0 to about 8.50 g of a.i./ha, or wherein the LC90 of the N,N-dimethyl-1,2,3-trithian-5-ylamine hydrochloride is between about 2.0 to about 30.00 g of a.i./mL at 100% purity of the N,N-dimethyl-1,2,3-trithian-5-ylamine hydrochloride.
